(19) United States
(12) Patent Application Publication
(10) Pub. No.: US 2014/0171890 A1
(43) Pub. Date: Jun. 19, 2014

(54) Antimicrobial Fabric and Method of Bactericidal Activation

(71) Applicant: R&T FABRIC, LLC, RIDGELAND, MS (US)

(72) Inventor: BERT H. RUBINSKY, JACKSON, MS (US)

(73) Assignee: R&T FABRIC, LLC, RIDGELAND, MS (US)

(21) Appl. No.: 13/947,080

(22) Filed: Jul. 21, 2013

Related U.S. Application Data
(63) Continuation-in-part of application No. 13/207,392, filed on Aug. 10, 2011, now Pat. No. 8,518,427.

(51) Int. Cl. A61L 15/18; A61L 15/46

(52) U.S. Cl. CPC A61L 15/18; A61L 15/46
USPC 604/360; 8/137

(57) ABSTRACT

A method of producing a fabric with antimicrobial properties that includes liquefying bamboo to produce a slurry, adding an antimicrobial element to the slurry, adding a non-bamboo fiber to the slurry to create a mixture and extruding the mixture to produce a fiber. The antimicrobial element may be silver particles that are ionized with a peroxide solution. The fabric may be incorporated into incontinence pads, garments and linens.

### CROSS-REFERENCE TO RELATED APPLICATIONS

This utility patent application filed under 35 USC §111 is a continuation-in-part of U.S. non-provisional application Ser. No. 13/207,392 filed on Aug. 10, 2011 and claims priority to U.S. provisional patent application Ser. No. 61,372,168 filed on Aug. 10, 2010, which is incorporated by reference herein.

### TECHNICAL FIELD

The technology relates to fabrics with antimicrobial properties, in particular, fabrics to reduce chances of infection.

### BACKGROUND

The spread of infectious disease through direct skin contact and open wounds is a well-known problem. In addition, the spread of “superbugs” such as methicillin-resistant Staphylococcus aureus (MRSA) has become a major problem in U.S.

MRSA can spread in hospitals and other health care facilities, and it can also be picked up in fitness centers, schools, and other public places. MRSA bacteria are resistant to most common antibiotics.

Hospitals typically take precautions to stop the spread of MRSA by stepping up infection control procedures. For example, hospitals implement various infection control procedures that include hand washing, skin disinfectants, sterilization, barrier protection, protective clothing and garments, sterile wound dressings and linen laundering procedures.

Despite these efforts at infection control, certain microbes and bacteria persist and continue to cause infection at an alarming rate. Thus, improvements in products and procedures to further inhibit the spread of infections are desired.

### SUMMARY

Antimicrobial and antibacterial textiles can play a part in a strategic plan to reduce healthcare associated infections. The present invention relates to the developments of fabrics, wraps and dressings that have improved antimicrobial/antimicrobial properties by combinations of bamboo, heavy metal and other fibers in the textile product.

The natural antibacterial/antimicrobial properties of bamboo fabric come from inherent qualities of bamboo. One of the compounds found in bamboo is coconut oil, which may contribute to the antimicrobial properties. Bamboo does not require the use of pesticides due to this natural antifungal antibacterial agent. It is rarely attacked by pests or infected by pathogens. The same natural substance that protects bamboo growing in the field, functions in the spun bamboo fibers.

In addition, the anti-infective activity of some heavy metals is well known. For example, heavy metals such as silver have been used as a topical therapy for burn wounds as an antiseptics or disinfectant. Inactivation of bacteria on surfaces containing silver and zinc ions has also been demonstrated.

In one general aspect, a method of producing a fabric with antimicrobial properties includes liquefying bamboo to produce a slurry, adding an antimicrobial element to the slurry, adding a non-bamboo fiber to the slurry to create a mixture and extruding the mixture to produce a fiber.

Embodiments may include one or more of the following features. For example, the method may include crushing the bamboo into fibers or small pieces. Liquefying the bamboo may include adding water and/or a solvent mixture.

The slurry may also be pressurized and/or heated to a high temperature. Impurities can also be removed from the slurry.

The antimicrobial element may be a heavy metal, such as, for example, silver. The silver may be silver ions or silver nanoparticles.

Extruding the mixture may be performed by passing the mixture through spinnerets to create the fiber. The fiber can also be spun to produce a thread and the thread weaved produce the antimicrobial fabric.

In another general aspect, an antimicrobial fabric includes bamboo fiber, a non-bamboo fiber and an antimicrobial element. The antimicrobial element may be a heavy metal such as silver and the non-bamboo fiber may cotton. The antimicrobial fabric may be composed of 69% bamboo, 30% cotton and 1% silver. Other natural and synthetic fibers may also be used.

In one general aspect, an incontinence pad, includes at least one antimicrobial layer, at least one fluid absorption layer, a waterproof barrier and a seam that attaches the at least one antimicrobial layer, the at least one fluid absorption layer, and the waterproof barrier.

Embodiments may include one or more of the following features. For example, the antimicrobial layer may be a fabric impregnated with one or more metal composition or metal ions having antimicrobial properties. The metal may be of a type that has antimicrobial effects, such as, ions of mercury, silver, copper, iron, lead, zinc, bismuth, gold, aluminum and other metals. More particularly, the metal may be silver particles, a silver compound, silver ions or nanoparticles that include silver. The antibacterial properties may be increased by ionizing the impregnated metal during fabrication or as part of a post-fabrication process.

As another feature, the antimicrobial layer may include a combination of natural fabrics such as cotton and bamboo. The fabric may also include synthetic materials. One or more metal, metal compound, metal composition or type of metal ion may be bonded to or attached to the fabric. The combination of fabric and metal may include, for example, a composition of 69% bamboo, 30% cotton and 1% silver by weight, respectively. In another embodiment, the fabric includes a combination with a range of approximately 10-95% bamboo, 5-90% other other type of natural or synthetic fiber and 0.001-5% metal by weight, respectively.

There can be more than one fluid absorption layer and more than one antimicrobial layer. For example, there may be two fluid absorption layers between the antimicrobial layer and the waterproof barrier. The fluid absorption layer may include natural or synthetic fabrics. For example, the fabric may be microfiber. However, other absorbent fabrics may be used.

In another embodiment, antimicrobial layers are also positioned between each fluid absorption layer.

The incontinence pad may be a number of different shapes, such as, for example, square or rectangular with rounded corners. It may have other configurations and may also be part of a wearable garment.

In another general aspect, a method of laundering a fabric that includes a metal to enhance bactericidal properties of the fabric includes immersing the fabric in water and adding a peroxide solution. Embodiments may include one or more of the following features.

The water may be at a temperature of 160° F. or more and the fabric may be immersed for at least six minutes.

The peroxide solution can be added so that the hydrogen peroxide is approximately 2% of a total volume of liquid.

As another feature, acid may be added to the water to produce hydrogen ions. A detergent may also be added to the water.

### DESCRIPTION OF THE DRAWINGS

FIG. 1 illustrates a method of producing an antimicrobial fabric;

FIGS. 2-6 are tables that provide test results for the antimicrobial fabric; and

FIGS. 7 and 8 are graphs that illustrate test results for the antimicrobial fabric.

FIGS. 9 and 10 illustrate the incontinence pad sketch.

FIG. 11 illustrates a method of enhancing antibacterial properties of the fabric of FIG. 1.

### DETAILED DESCRIPTION

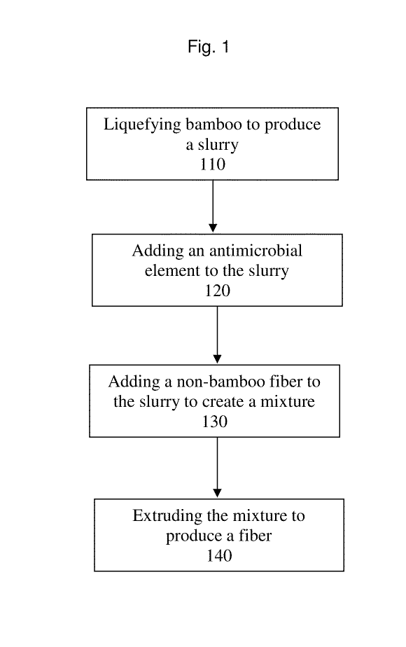

In one embodiment, a textile with antimicrobial properties is produced by a process described with respect to FIG. 1. First, the bamboo is liquefied into a slurry (operation 110). Typically, the bamboo is crushed into fibers or small pieces. The slurry may be produced by, for example, using water and a solvent mixture. The water and solvents may be added in a vat that is pressurized and heated until the bamboo fibers dissolve.

Impurities are then removed from the slurry. Silver is added to the liquefied bamboo (operation 120). The silver may be particles or silver nanoparticles that are added to the slurry. Silver oxide or silver ions may also be used. In other embodiments, other heavy metals may be used such as copper.

Then, cotton is added to the mixture (operation 130). Other fibers may be added to increase the absorption of silver particles into the fibers.

The composite is then woven into fabric (operation 140). For example, it may be extruded through spinnerets to create a thin and strong fiber. The fibers are then spun into a thread and weaved into the fabric.

The fabric may be further processed such as, for example, dyed to a desired color or cut to size for use in sheets or bandages.

In one of the embodiments of the novel material, the fabric is approximately 69% bamboo, 30% cotton and 1% silver. Other combinations of bamboo, cotton and an antimicrobial element such as a heavy metal may be used. For example, the composition may be 49% bamboo, 50% cotton and 1% silver. In other embodiments, synthetic fibers, such as spandex, may be added to the fabric.

Test reports have demonstrated that the fabric made of bamboo, cotton and silver has improved antimicrobial properties.

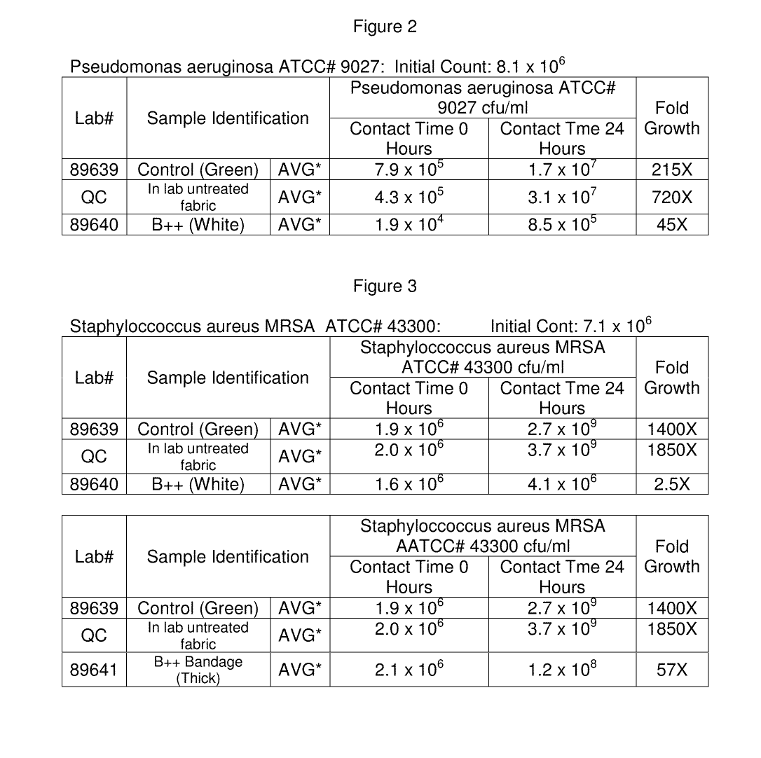

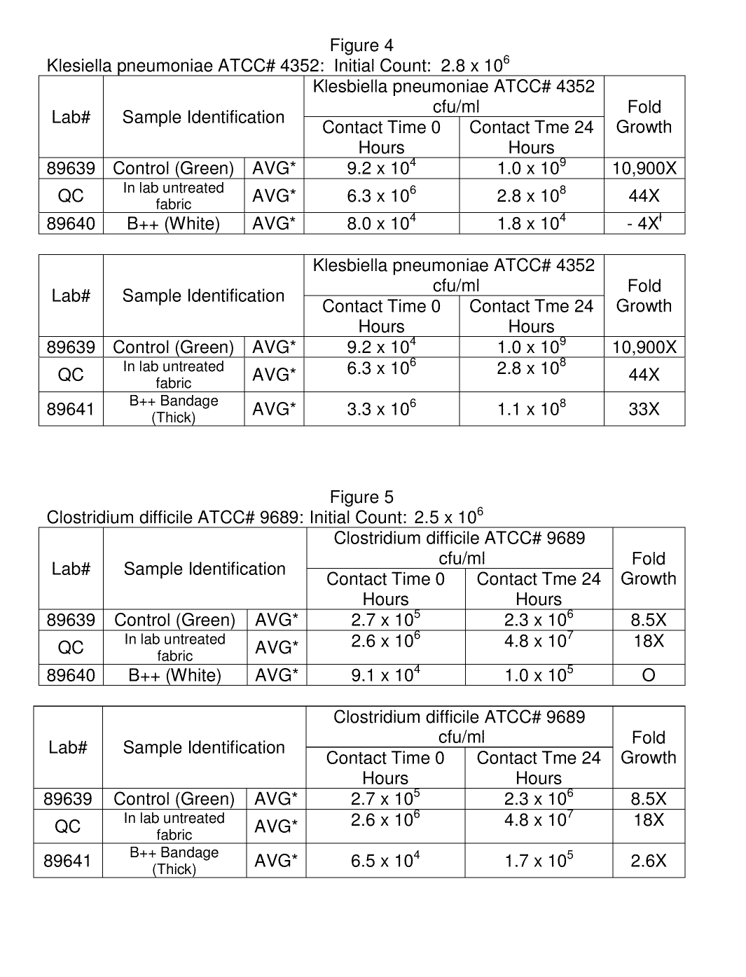

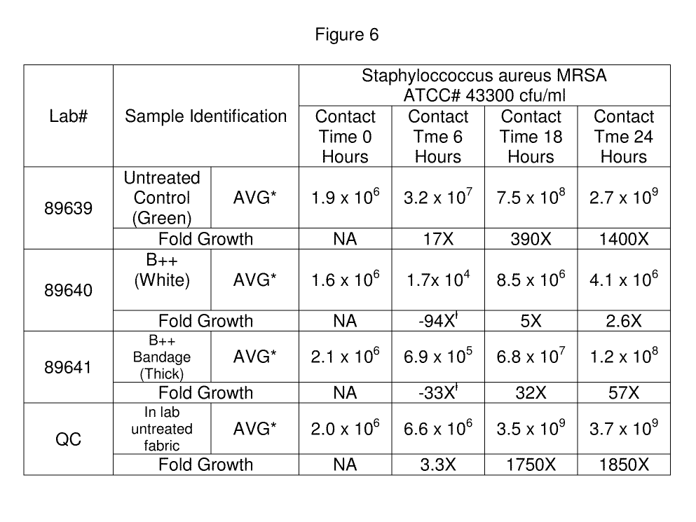

FIGS. 2-6 are tables that illustrate test results showing the antibacterial properties of the novel material as compared to other fabrics. Swatches of treated and untreated fabric were cut into 4.8 cm diameter discs which were inoculated with 1 ml of a test organism in a concentration of 1-2×105. Each stack was aseptically transferred to sterile screw cap jars and incubated at 35° C. Treated and untreated samples with no inoculums were also set up as control. After specified time period the set of treated and untreated swatches were removed from the incubator and were neutralized with 100 ml of a neutralizer. Plate counts were performed and incubation was carried out according to requirements for each organism.

Percent reduction of bacteria was calculated by the following formulas:

% R=100(B−A)/B where: R: % reduction
A: the number of bacteria recovered from the inoculated treated test specimen swatches in the jar incubated over the desired contact period
B: the number of bacteria recovered from the inoculated treated test specimen swatches in the jar immediately after inoculation (at “0” contact time).

The data is presented in Colony Forming Units (CFU)/ml after control and test samples were exposed to organisms. In all cases, the novel material (B++) showed significant inhibition of the four bacterial species tested at 24 hours after inoculation. Additionally, the novel materials showed significant antimicrobial activity at 6 hours after inoculation and two orders of magnitude inhibition at 18 and 24 hours against Staphylococcus Aureus MRSA. These results strongly suggest that both the B++ and the treated B++ Bandage could play a significant role in reducing the rate of nosocomial infections.

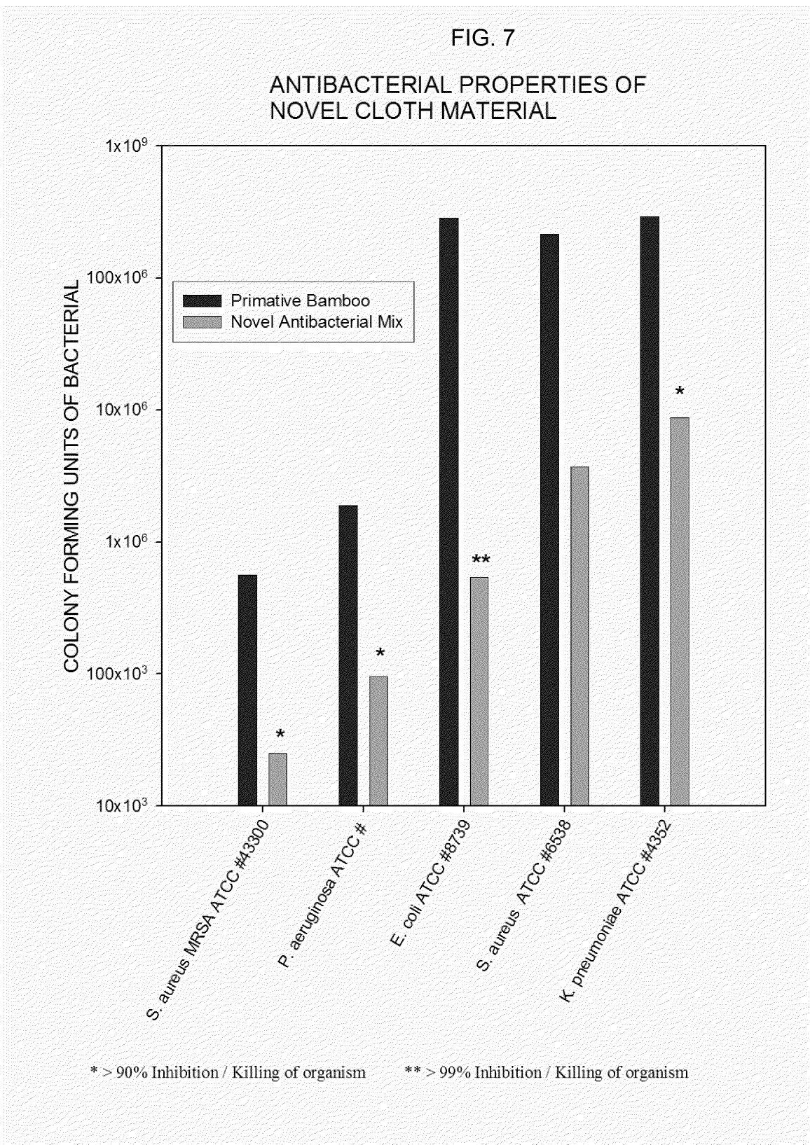

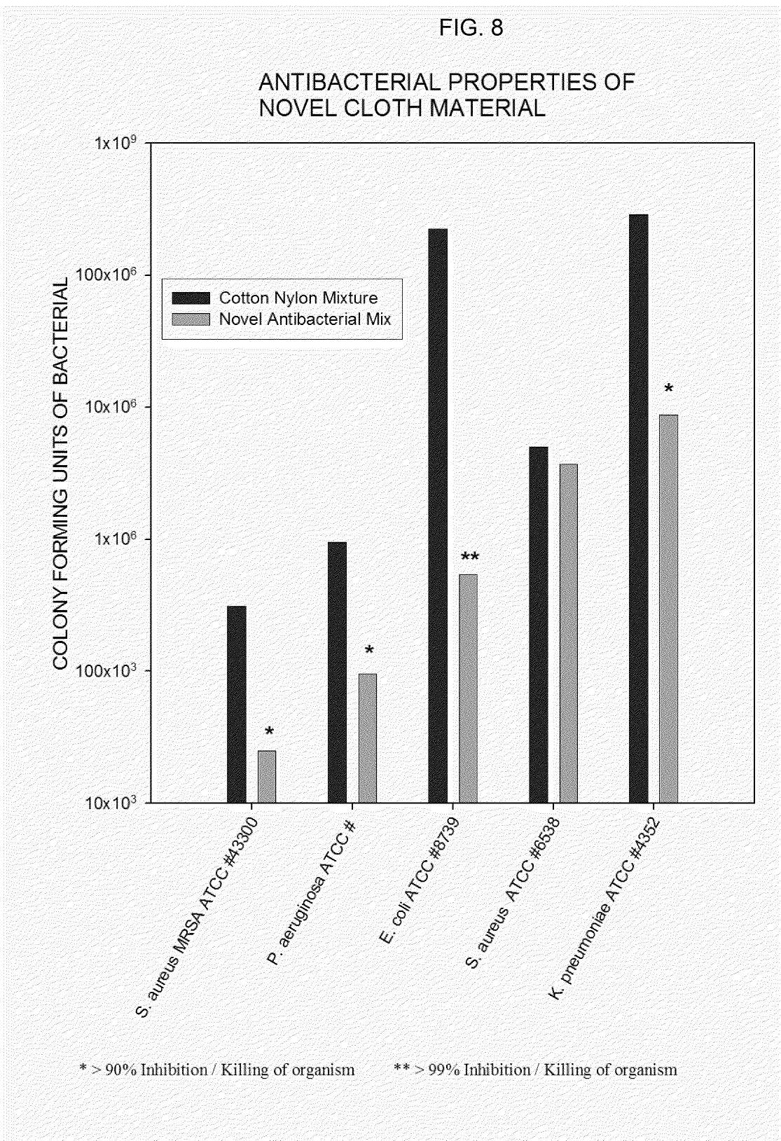

Another test result is shown graphically in FIGS. 7 and 8. The results shown in FIG. 7 are a comparison of the novel material to bamboo material and cotton-nylon mixtures respectively.

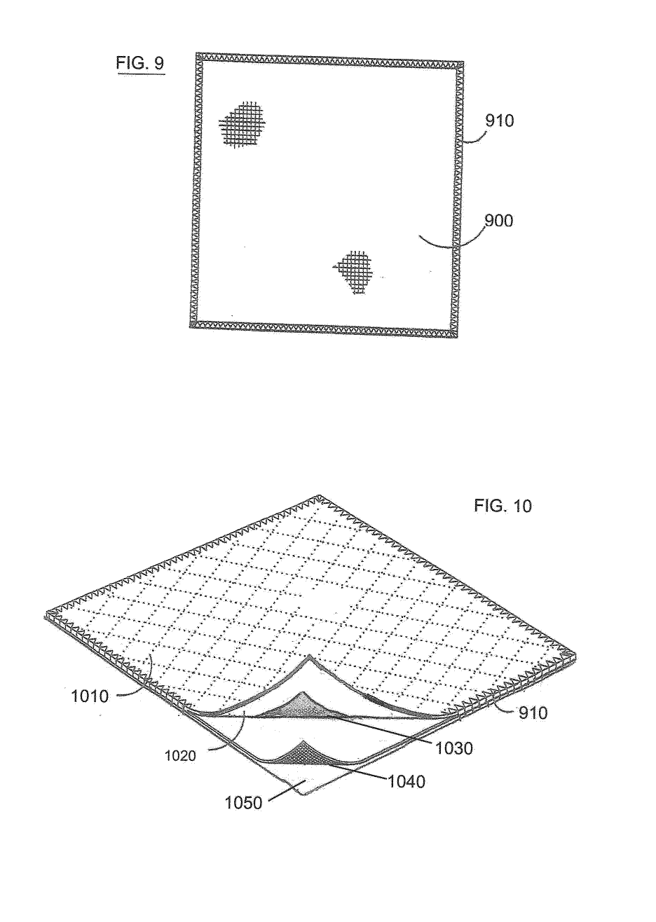

FIGS. 9 and 10 show the antibacterial fabric configured as an incontinence pad 900. The incontinence pad is made of multiple fabric layers and the edges are attached by a seam 910.

In the embodiment shown in FIG. 10, the incontinence pad has four layers. The top layer is an antimicrobial layer 1010. The antimicrobial layer 1010 can be made of the antimicrobial fabric mentioned above. The antimicrobial layer is permeable to fluid so that any fluid passes through the bottom 1020 to the layers below.

The next layers are first and second absorbent layers 1030, 1040. The absorbent layers 1030, 1040 are made of microfiber which traps and holds moisture. In other embodiments, the layers can be made of terrycloth, woven cotton, acrylic or other mixtures of synthetic and natural fibers.

The absorbent layers, 1030, 1040, may include various other types of nonwoven fabrics. Nonwoven fabric is a fabric-like material made from long fibers, bonded together by chemical, mechanical, heat or solvent treatment. This includes fabric such as felt, which is neither woven nor knitted. Nonwoven materials typically lack strength unless densified or reinforced by a backing. In recent years, nonwovens have become an alternative to polyurethane foam. Absorbency rate and absorbent capacity are the two most important performance parameters to be considered for selection of material. The absorbent capacity is mainly determined by the interstitial space between the fibers, the absorbing and swelling characteristics of the material and the resiliency of the web in the wet state. The absorbency rate is governed by the balance between the forces exerted by the capillaries and the frictional drag offered by the fiber surfaces.

For non-swelling materials, these properties are largely controlled by the capillary sorption of fluid into the structure until saturation is reached. The absorbency rate and absorbent capacity are affected by fiber mechanical and surface properties, structure of the fabric, such as, for example, the size and the orientation of flow channels, and the nature of fluids imbibed. Among those factors, the surface wetting characteristics (contact angle) of the fibers in the web and the structure of the web, such as the size, shape, orientation of capillaries, and the extent of bonding, are significant.

The polymer type of the fibers in the fabrics, hydrophilic or hydrophobic, influences the inherent absorbent properties of the fabrics. A hydrophilic fiber provides the capacity to absorb liquid via fiber imbibitions, giving rise to fiber swelling. It also attracts and holds liquid external to the fiber, in the capillaries, and structure voids. On the other hand, a hydrophobic fiber has only the latter mechanism available to it normally [7]. The effect of the small amount of fiber finish (generally 0.1 to 0.5% by weight) is also important since it is on the fiber surface. The particular finish applied on the fiber can significantly change surface wetting property of the fiber.

Fiber linear density and its cross-section area affect void volume, capillary dimensions and the total number of capillaries per unit mass in the fabrics. Fiber surface morphology, surface ruggedness, and core uniformity can influence the absorbency performance to some extent. Fiber crimps influence the packing density of the fabrics and further affect the thickness per unit mass that affects the absorbency of the nonwoven fabrics. The nature of the crimps, whether it is two-dimensional or three-dimensional, also has some effect.

The size of capillaries is affected by the thickness per unit mass and the resiliency of the web, and the size, shape and the mechanical properties of the fibers. The resiliency of the web is influenced by the nature and level of bonding of the fabrics as well as the size, shape, and mechanical properties of the constituent fibers.

The bottom layer is a waterproof layer 1050. The waterproof layer may incorportate waterproof fabric that inherently, or has been treated to become, resistant to penetration by water and wetting. It can be made of natural or synthetic fabrics that are laminated to or coated with a waterproofing material such as rubber, polyvinyl chloride (PVC), polyurethane (PU), silicone elastomer, fluoropolymers, and/or wax. Other examples include rubberised fabric used in sauna suits and inflatable boats.

If the incontinence pad 900 is configured as, for example, a diaper, the waterproof fabric may be breathable to resist liquid water passing through, but allowing water vapor to pass through.

In use, the incontinence pad 900 may be placed on, for example, a hospital bed. The incontinence pad 900 is positioned between the mattress and the patient with the antibacterial layer 1010 facing the patient and the waterproof layer 1050 facing down toward the bed. If fluids are released on the incontinence pad 900, the fluid passes through the antibacterial layer 1010 and is absorbed by the absorbent layers 1030, 1040. The waterproof layer 1050 prevents the fluid from seeping into the mattress.

Since the antibacterial layer 1010 has active bactericidal properties, the incontinence pad 900 can reduce the risks of infection caused by bed sores and/or a moist environment. In other words, bacteria in the absorbent layers 1030, 1040 is not transmitted back to the patient.

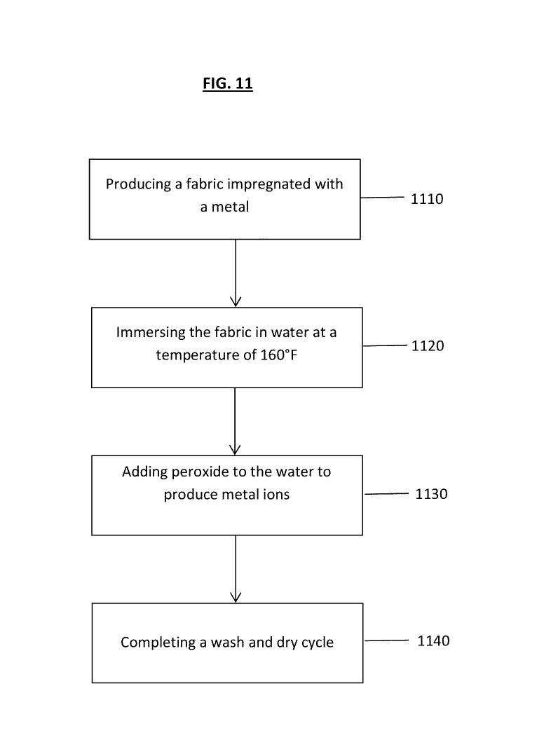

FIG. 11 illustrates a method of laundering a fabric impregnated with a metal element that enhances bactericidal properties of the fabric. In operation 1110, a fabric is impregnated with a metal, such as, for example, silver, silver oxide or silver nanoparticles. If silver particles are used, the fabric may be impregnated with more than 600 milligrams of silver per ounce of fabric. If nanoparticles are impregnated into the fabric, the silver content may be about 75 mg per ounce of fabric.

In operation 1120, the fabric is placed in a tub of a washing machine and water is added to the tub. During the wash process, the water may be at a temperature of at least 160° F. and the fabric may be immersed for at least six minutes. This temperature and immersion time should kill most pathogens.

In operation 1130, hydrogen peroxide is added to the water. In one embodiment, the hydrogen peroxide is added at a concentration of 35% by volume. The total peroxide that is added may be approximately 2 oz. per each 100 lbs in weight of fabric. As described in more detail below, the amount of peroxide can be adjusted depending on the desired concentration and pH levels to produce optimal results.

Note that these operations may be completed in a different order. For example, the hydrogen peroxide may be added to the tub and mixed with the water prior to adding the fabric.

As explained in more detail above, the fabric is impregnated with a heavy metal such as, for example, silver particles, and it may also be a mix of materials such as cotton and bamboo. Bamboo is known to have antimicrobial properties. In addition, fibrous or absorbent materials may have more capacity to hold silver particles. In other embodiments, the silver is chemically bonded to the fabric and/or silver nanoparticles are used.

As the total amount of hydrogen peroxide is added, the pH drops as the solution becomes more acidic as shown in Table 1.

TABLE 1

| | H₂O₂ Conc. | | | | | | | | | | |
|---|---|---|---|---|---|---|---|---|---|---|---|
| | 0 | 10 | 20 | 30 | 40 | 50 | 60 | 70 | 80 | 90 | 100 |
| pH @ 25° C. | 7.0 | 5.3 | 4.9 | 4.7 | 4.6 | 4.5 | 4.5 | 4.5 | 4.6 | 4.9 | 6.2 |

When hydrogen peroxide is added, hydrogen ions are formed. A reaction is represented as follows:

2Ag+H₂O₂(aq)+2H⁺(aq) [symbol] 2Ag⁺+2H₂O

where Ag is silver, H₂O₂ is hydrogen peroxide, H⁺ is hydrogen ions and H₂O is water. Thus, the silver becomes a silver ion with more active bactericidal properties. The metal ions have a more toxic effect on living cells, algae, molds, spores, fungi, viruses, prokaryotic and eukaryotic microorganisms, even in relatively low concentrations. Other types of metals, such as copper or gold, may be ionized to produce more active antimicrobial properties.

In some cases, hydronium ions (H₃O⁺) react with the silver (Ag) to produce the silver ions. In certain cases, a metal oxide, such as, for example, silver oxide, may be involved in the process of reducing bacteria.

As another feature, an acid may be added to the water to produce additional hydrogen ions. In operation 1140 a wash and rinse cycle is completed. For example, a detergent may be added to the water and the tub may be agitated. The fabric is then dried and is ready for use.

Since certain changes may be made in the above process without departing from the scope of the invention herein involved, it is intended that all matter contained in the above description or shown in the accompanying drawing shall be interpreted in an illustrative and not in a limiting sense. Accordingly, other implementations are within the scope of the following claims.

1. An incontinence pad, comprising:
at least one antimicrobial layer;
at least one fluid absorption layer;
a waterproof barrier; and
a bond that attaches the at least one antimicrobial layer, the at least one fluid absorption layer, and the waterproof barrier.

2. The incontinence pad of claim 1, wherein the at least one antimicrobial layer comprises a fabric impregnated with a metal, metal composition, metal compound and/or metal ions having antimicrobial properties.

3. The incontinence pad of claim 1, wherein the at least one antimicrobial layer comprises a fabric impregnated with silver particles and silver ions.

4. The incontinence pad of claim 1, wherein the at least one antimicrobial layer comprises a fabric impregnated with a silver compound.

5. The incontinence pad of claim 1, wherein the at least one antimicrobial layer comprises a fabric impregnated with silver in a range of 10-1,000 milligrams of silver per ounce of fabric.

6. The incontinence pad of claim 1, wherein the at least one antimicrobial layer comprises a fabric that includes bamboo and cotton.

7. The incontinence pad of claim 1, wherein the at least one antimicrobial layer comprises a fabric that includes cotton, bamboo and metal.

8. The incontinence pad of claim 1, wherein the at least one antimicrobial layer comprises a composition of 69% bamboo, 30% cotton and 1% silver.

9. The incontinence pad of claim 1, wherein the at least one fluid absorption layer comprises a first fluid absorption layer and a second fluid absorption layer.

10. The incontinence pad of claim 1, wherein the at least one fluid absorption layer comprises microfiber.

11. The incontinence pad of claim 1, wherein the incontinence pad comprises a rectangular shape.

12. The incontinence pad of claim 1, wherein the incontinence pad comprises a wearable garment.

13. The incontinence pad of claim 1, wherein the bond comprises a seam along an outside perimeter or outer edge of each layer to attach the least one antimicrobial layer, the at least one fluid absorption layer, and the waterproof barrier.

14. A method of laundering a fabric that includes a metal to enhance bactericidal properties of the fabric, the method comprising:
immersing the fabric in water; and
adding a peroxide solution.

15. The method of claim 14, wherein immersing the fabric in water comprises immersing the fabric in hot water at a temperature of 160 degrees Fahrenheit for at least six minutes.

16. The method of claim 14, wherein adding a peroxide solution comprises adding hydrogen peroxide.

17. The method of claim 14, wherein adding the peroxide solution comprises adding hydrogen peroxide so that the hydrogen peroxide comprises approximately 2% of a total volume of liquid.

18. The method of claim 14, adding hydrogen ions to the water.

19. The method of claim 14, further comprising adding an acid to the water to produce hydronium ions.

20. The method of claim 14, further comprising adding a detergent to the water.
